(19) United States
(12) Patent Application Publication
(10) Pub. No.: US 2024/0077412 A1
(43) Pub. Date: Mar. 7, 2024

(54) OPTICAL IMAGING APPARATUS, PROCESSING APPARATUS, OPTICAL IMAGING METHOD, AND NON-TRANSITORY STORAGE MEDIUM

(71) Applicant: KABUSHIKI KAISHA TOSHIBA, Tokyo (JP)

(72) Inventors: Hiroya KANO, Kawasaki Kanagawa (JP); Hiroshi OHNO, Tokyo (JP); Kenta TAKANASHI, Kawasaki Kanagawa (JP)

(73) Assignee: KABUSHIKI KAISHA TOSHIBA, Tokyo (JP)

(21) Appl. No.: 18/174,684

(22) Filed: Feb. 27, 2023

(30) Foreign Application Priority Data
Sep. 6, 2022 (JP) 2022-141492

(51) Int. Cl. G01N 21/31; G06T 7/90; H04N 23/10; H04N 23/56

(52) U.S. Cl. CPC G01N 21/31; G06T 7/90; H04N 23/10; H04N 23/56; G06T 2207/10024

(57) ABSTRACT

According to the embodiment, an optical imaging apparatus includes: an illuminator, a lens, an aperture, and an imaging element. Light is incident into the lens through an inspection object provided where the parallel light from the illuminator reaches. The light has passed through the solvent and the target, and/or through the solvent. The aperture is disposed on a focal plane of the lens. The aperture includes a passage region and a light-blocking region. The passage region allows passage of diffracted light in a direction different from a direction of the parallel light due to the target from the parallel light from the illuminator. The light-blocking region blocks the parallel light having passed through the solvent.

### CROSS-REFERENCE TO RELATED APPLICATIONS

This application is based upon and claims the benefit of priority from Japanese Patent Application No. 2022-141492, filed Sep. 6, 2022, the entire contents of all of which are incorporated herein by reference.

### FIELD

Embodiments described herein relate generally to an optical imaging apparatus, a processing apparatus, an optical imaging method, and a non-transitory storage medium.

### BACKGROUND

For example, in order to measure the shape, size, or physical properties of a target in solvent, optical techniques are key.

### BRIEF DESCRIPTION OF THE DRAWINGS

FIG. 1 is a schematic view of an optical imaging apparatus according to an embodiment.

FIG. 2 illustrates an aperture along line II-II in the optical imaging apparatus illustrated in FIG. 1.

FIG. 3 is an explanatory view for diffraction of light in the optical imaging apparatus illustrated in FIG. 1.

FIG. 4 illustrates an exemplary image captured with the optical imaging apparatus illustrated in FIG. 1.

FIG. 5 illustrates an exemplary image captured with the optical imaging apparatus, illustrated in FIG. 1, from which the aperture is removed.

FIG. 6 illustrates, on its left side, an exemplary image captured with a typical optical imaging apparatus and illustrates, on its right side, an exemplary image captured with a Schlieren method, in which the subjects are the same.

FIG. 7 is a schematic view of a water treatment system including the optical imaging apparatus according to the embodiment.

FIG. 8 is a schematic view of a floc in water being treated in FIG. 7.

FIG. 9 illustrates an exemplary image captured with the angle θ of diffraction ranging from 0 to 0.14° with the optical imaging apparatus applied to the water treatment system illustrated in FIG. 7.

FIG. 10 illustrates an exemplary image captured with the angle θ of diffraction ranging from 0.14 to 0.42° with the optical imaging apparatus applied to the water treatment system illustrated in FIG. 7.

FIG. 11 illustrates an exemplary image captured with the angle θ of diffraction ranging from 0.42 to 0.70° with the optical imaging apparatus applied to the water treatment system illustrated in FIG. 7.

FIG. 12 illustrates an exemplary image captured with the optical imaging apparatus, from which the aperture is removed, applied to the water treatment system illustrated in FIG. 7.

FIG. 13 illustrates exemplary hue histograms generated from color images acquired with the optical imaging apparatus applied to the water treatment system illustrated in FIG. 7.

FIG. 14 is a graph of which the horizontal axis represents the concentration ratio ρ of a floc and the vertical axis represents the ratio γ between the average near the first peak and the average near the second peak acquired from each hue histogram, with the optical imaging apparatus applied to the water treatment system illustrated in FIG. 7.

FIG. 15 is a flowchart describing a flow of processing of estimating information related to a physical property of a target, with the optical imaging apparatus.

### DETAILED DESCRIPTION

An object of the present invention is to provide an optical imaging apparatus, a processing apparatus, an optical imaging method, and a non-transitory storage medium storing an optical imaging program that enable noncontact acquisition of information according to a target in solvent.

According to the embodiment, an optical imaging apparatus includes: an illuminator, a lens, an aperture, and an imaging element. The illuminator is configured to emit parallel light including at least two or more different wavelength spectra of light. Light is incident into the lens through an inspection object provided where the parallel light from the illuminator reaches. The inspection object includes a solvent and a target in the solvent serving as a substance different from the solvent. The light has passed through the solvent and the target, and/or through the solvent. The aperture is disposed on a focal plane of the lens. The aperture includes a passage region and a light-blocking region. The passage region allows passage of diffracted light in a direction different from a direction of the parallel light due to the target from the parallel light from the illuminator. The light-blocking region blocks the parallel light having passed through the solvent. The imaging element is configured to acquire, at each pixel, in response to arrival of the diffracted light based on diffraction due to the target at an imaging plane through the passage region, the at least two or more different wavelength spectra of light, simultaneously and distinctively.

Embodiments will be described below with reference to the drawings. The drawings are schematic or conceptual, and thus, for example, the relationship between the thickness and width of each part or the ratio in size between parts is not necessarily identical to a reality. In some cases, the same parts between drawings are different in dimensions or ratio. In the present specification and the figures, elements similar to those described regarding already mentioned figures are denoted with the same reference signs and thus detailed descriptions thereof will be appropriately omitted.

Light or a beam in each embodiment is not limited to visible light or a visible beam. Note that, in the following description, white light is exemplarily used.

An optical imaging apparatus 10 according to the present embodiment will be described with reference to the drawings.

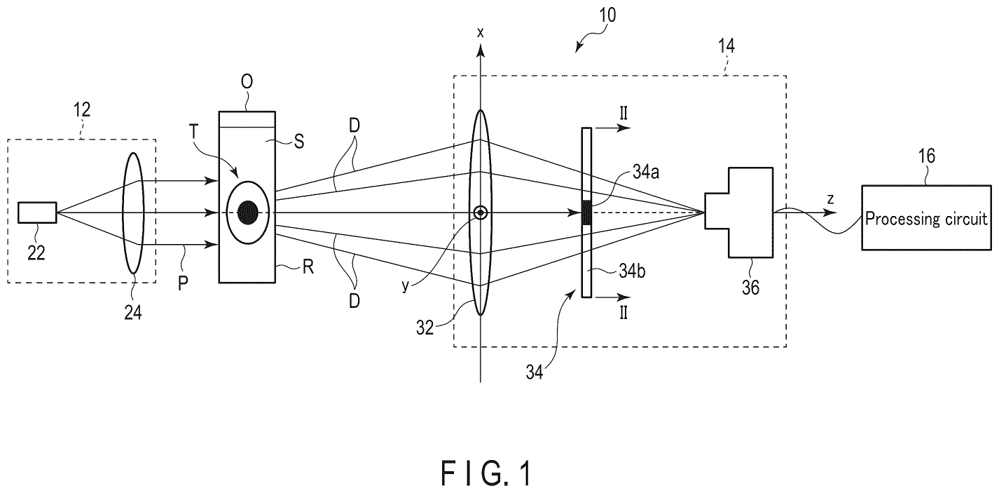

FIG. 1 is an x-z sectional view of the optical imaging apparatus 10 according to the present embodiment. The optical imaging apparatus 10 includes an illuminator 12 that illuminates an inspection object O, an imaging portion 14 that selectively acquires light through the inspection object O, and a processing circuit (processing apparatus) 16 connected to the imaging portion 14.

In the present embodiment, x, y, and z axes are each defined as follows. The x and y axes are mutually orthogonal and are each orthogonal to the z axis.

The z axis corresponds to the optical axis of the illuminator 12 and passes through the centers of the illuminator 12 and the imaging portion 14 as optical elements included in the optical imaging apparatus 10. The +z direction corresponds to the direction from the illuminator 12 to an imaging element 36. The −x direction corresponds, for example, to the direction of gravity. Here, for example, referring to the x-z sectional view of the optical imaging apparatus 10 illustrated in FIG. 1, the +z direction corresponds to the direction from left to right, the −x direction corresponds to the direction from above downward, and the +y direction corresponds to the direction from back to front, perpendicularly to the x-z sectional view.

The illuminator 12 is allowed to have an appropriate structure, provided that two or more wavelength spectra of light can be emitted as parallel light P to the inspection object O and the imaging portion 14. In the present embodiment, the illuminator 12 includes a light source 22 and an illumination lens 24. Such parallel light has its beams in parallel. In the present embodiment, such beams are required to be substantially in parallel. For example, in a case where the focal length and diameter of a lens are defined as f and d, respectively, the angle of divergence of light is required to be not more than tan⁻¹(d/f).

The light source 22 of the illuminator 12 is, for example, a light-emitting diode (LED) and emits white light including red (R) light, green (G) light, and blue (B) light. In the present embodiment, the light source 22 measures, for example, 100 μm per side. The light source 22 is not limited to such an LED and thus may be, for example, an incandescent lamp, a fluorescent tube, or a mercury lamp. The light source 22 is not limited to white-light emission. Light to be emitted from the light source 22 is required to include two or more different wavelength spectra of light. Such two or more different wavelength spectra of light correspond, for example, to blue light having a wavelength of 450 nm (first wavelength) and red light having a wavelength of 650 nm (second wavelength), namely, appropriate light having different peak wavelengths. The light source 22 may emit a plurality of wavelength spectra of light different from such wavelength spectra of light as above.

The illumination lens 24 makes the light from the light source 22 the parallel light P such that the parallel light P is emitted to the inspection object O and the imaging portion 14. The illumination lens 24 may be a single lens or a compound lens including a plurality of lenses in combination. As the illumination lens 24, for example, a collimator lens is used. In the present embodiment, the focal length of the illumination lens 24 of the optical imaging apparatus 10 is 200 mm.

The imaging portion 14 includes a lens (imaging lens) 32, an aperture 34, and the imaging element 36.

The lens 32 focuses on the image side. The inspection object O is disposed between the lens 32 and the illumination lens 24 of the illuminator 12. The inspection object O is provided where the parallel light emitted from the illuminator 12 reaches the inspection object O. Thus, from the illuminator 12, diffracted light D, to be described below, having passed through solvent S and a target T and/or the parallel light P having passed through the solvent S are incident into the lens 32. The lens 32 may be a single lens or a compound lens including a plurality of lenses in combination. In the optical imaging apparatus 10 according to the present embodiment, rightfully, the light from the light source 22 is allowed to pass in air and/or a vacuum different from the solvent S of the inspection object O. In the present embodiment, the focal length f of the imaging lens 32 of the optical imaging apparatus 10 is 200 mm.

The aperture 34 is provided on the focal plane on the image side of the lens 32. The aperture 34 includes a light-blocking region 34a located on the optical axis L of the lens 32 and a passage region 34b outside the light-blocking region 34a.

The light-blocking region 34a is disposed at the focal position of the lens 32. Thus, when the light through the lens 32 to the light-blocking region 34a is incident into the light-blocking region 34a, the light-blocking region 34a blocks and prevents the incident light from travelling to the imaging element 36. Preferably, the light-blocking region 34a is provided axisymmetrically about the optical axis L (z axis). The light-blocking region 34a according to the present embodiment is, for example, circular in shape, resulting in being axisymmetric about the optical axis L (z axis).

The size of the light-blocking region 34a is adjusted based on the light source 22 of the illuminator 12 and the optical magnification of the lens 32 disposed on the optical path from the light source 22 to the aperture 34. The lower limit of the size of the light-blocking region 34a corresponds to the value of multiplication of the light source 22 of the illuminator 12 and the optical magnification of the lens 32. Thus, if the respective optical magnifications of the lens 32 and the illumination lens 24 are onefold, the size of the light-blocking region 34a is identical to or larger than the size of the light source 22.

In other words, the optical imaging apparatus 10 serves as an optical system in which the light from the light source 22 is projected on the light-blocking region 34a. The optical imaging apparatus 10 according to the present embodiment blocks, with the light-blocking region 34a, the light from the light source 22. Therefore, the light-blocking region 34a is identical to or larger than the size of projection of the light from the light source 22.

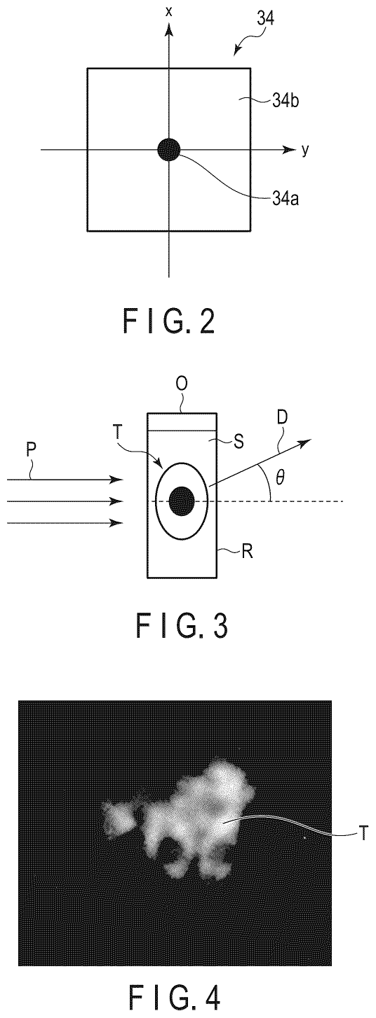

In the present embodiment, the light-blocking region 34a of the aperture 34 is circular in shape and 0.5 mm in diameter to the target T. FIG. 2 illustrates an exemplary external appearance of the aperture 34 used in practice. The aperture 34 is formed by forming a carbon with a diameter of 0.5 mm as the light-blocking region 34a and embedding the carbon in a thin and transparent glass plate with a thickness of 0.5 mm. Note that the size of the light-blocking region 34a is likely to vary from the lower limit described above, depending on the solvent S of the inspection object O and the target T in the solvent S.

The passage region 34b allows light to pass therethrough. For example, the passage region 34b is formed of a transparent glass plate having an appropriate thickness. Preferably, the passage region 34b that barely affects light to pass through the passage region 34b is selected.

Note that the external shape of the aperture 34, namely, the external shape of the passage region 34b is appropriately formed, for example, like a circle or a rectangle. As illustrated in FIG. 2, in the present embodiment, the aperture 34 has the light-blocking region 34a circular at its center (its centroid) and the passage region 34b, of which the external shape is rectangular, outside the light-blocking region 34a.

A plane including the region in which the imaging element 36 is disposed is defined as the image plane (imaging plane) of the lens 32. The imaging element 36 is achieved with an area sensor. Such an area sensor includes pixels areally arrayed in the same plane. The imaging element 36 according to the present embodiment includes a plurality of pixels. Used is a so-called RGB camera in which each pixel is capable of receiving at least two different wavelength spectra of beams, namely, a first wavelength spectrum of beam and a second wavelength spectrum of beam different from the first wavelength spectrum of beam. Preferably, each pixel of the imaging element 36 has color channels enabling distinctive reception of a plurality of predetermined wavelength spectra of light, like three channels for R, G, and B. Note that respective independent pixels to R, G, and B may be provided and the respective pixels of R, G, and B may be collectively regarded as a single pixel. In the present embodiment, each pixel of the imaging element 36 has at least two color channels for red (R) and blue (B). Thus, the imaging element 36 can receive, at each pixel, blue light having a wavelength of 450 nm and red light having a wavelength of 650 nm through the respective independent color channels. The imaging element 36 according to the present embodiment can receive, for example, green light having a wavelength of 550 nm through an independent color channel.

The imaging element 36 can be achieved, for example, with a charge-coupled device (CCD). The imaging element 36 may be achieved, for example, with a 1CCD color CCD or a 3CCD color CCD. The imaging element 36 is not limited to such CCDs and thus may be an image capturing sensor, such as a complementary metal-oxide semiconductor (CMOS) or a light-receiving element.

The processing circuit 16 controls light emission/non-light emission of the light source 22 and additionally controls the imaging element 36 to acquire an image during light emission of the light source 22. In the present embodiment, the processing circuit 16 controls the imaging element 36 and additionally performs various types of computing to image data acquired from the imaging element 36.

The processing circuit 16 acquires, as an image, light having entered the imaging plane of the imaging element 36 and additionally outputs the intensity of received light of each color channel for each pixel. That is, the processing circuit 16 outputs the intensity of received light at the position of reception of a beam having entered the imaging plane of the imaging element 36. Hereinafter, data acquired by the processing circuit 16 with the imaging element 36 is referred to as an image.

The processing circuit 16 is achieved, for example, with a computer and includes a processor (processing circuit) and a storage medium. As the processor, provided is any of a central processing unit (CPU), an application specific integrated circuit (ASIC), a microcomputer, a field programmable gate array (FPGA), and a digital signal processor (DSP). As the storage medium, provided can be an auxiliary storage device, in addition to a main storage device, such as a memory. Examples of the storage medium include nonvolatile memories enabling writing and reading at any time, such as a hard disk drive (HDD), a solid state drive (SSD), a magnetic disk, an optical disc (e.g., a CD-ROM, a CD-R, or a DVD), a magneto-optical disc (e.g., an MO), and a semiconductor memory.

The numbers of processors and storage media to be provided in the processing circuit 16 may be each one or two or more. In the processing circuit 16, for example, the processor executes a program stored in the storage medium to perform processing. The program to be executed by the processor of the processing circuit 16 may be stored in a computer (server) or a server under cloud computing connected to the processing circuit 16 through a network, such as the Internet. In this case, the processor downloads the program through the network. In the processing circuit 16, for example, the processor performs ON/OFF of the light source 22, image acquisition from the imaging element 36, and various types of calculation processing based on an image acquired from the imaging element 36, and the storage medium functions as a data storage.

At least part of processing in the processing circuit 16 may be performed by a cloud server under cloud computing. An infrastructure of cloud computing is achieved with a virtual processor, such as a virtual CPU, and a cloud memory. In an example, a virtual processor performs ON/OFF of the light source 22, image acquisition from the imaging element 36, and various types of calculation processing based on an image acquired from the imaging element 36, and a cloud memory functions as a data storage.

Note that, in the present embodiment, the inspection object O including the solvent S and the target T is disposed between the illuminator 12 and the lens 32. In the present embodiment, the inspection object O has the target T in the solvent S. The solvent S is a liquid and is, for example, water. The solvent S may be material different from water. The solvent S may be colored. Used is the solvent S that allows the parallel light P of light emitted from the light source 22 of the illuminator 12 to pass through without refraction.

Here, the function of the optical imaging apparatus 10 to acquire an image of the inspection object O will be described.

When the processing circuit 16 acquires an image with the imaging element 36, the processing circuit 16 causes light emission of the light source 22 of the illuminator 12, so that the parallel light P parallel to the optical axis L is emitted from the illuminator 12. The solvent S and the target T in a receptacle R for the inspection object O are irradiated with the parallel light P from the illuminator 12. The receptacle R and the solvent S illustrated in FIG. 1 are required to allow illuminating light from the illuminator 12 to pass through. When the inspection object O is irradiated with the parallel light P, the parallel light P having passed through the receptacle R and the solvent S without irradiating the target T reaches, through the lens 32, the light-blocking region 34a of the aperture 34 at the focal point of the lens 32. The size of the light-blocking region 34a is larger than the size of projection of the light from the light source 22 onto the light-blocking region 34a. Thus, the parallel light P is blocked by the light-blocking region 34a and thus does not reach the imaging element 36.

As illustrated in FIG. 3, when the inspection object O is irradiated with the parallel light, the target T in the solvent S is irradiated with the parallel light. At this time, if the size of the structure of the target T in the solvent S is approximately the same as component included in wavelengths of the parallel light, when the target S is irradiated with the parallel light, part of the parallel light is diffracted by the target T. Such diffraction can occur even in a case where the target T is transparent.

As illustrated in FIG. 1, the diffracted light D, having deviated from the z axis (optical axis) along the parallel light P, due to diffraction of light, travels to the lens 32. The diffracted light D which has been incident into the lens 32 is incident into the imaging plane of the imaging element 36 through the lens 32. In this case, the diffracted light D through the lens 32 passes through the passage region 34b out of the light-blocking region 34a on the z axis of the aperture 34. The passage region 34b is disposed on the focal plane of the lens 32 and allows passage of the diffracted light D in a direction different from the direction of the parallel light P due to the target T from the parallel light P from the illuminator 12. In response to arrival of the diffracted light D based on diffraction due to the target T at the imaging plane of the imaging element 36 through the passage region 34b, the imaging element 36 acquires, at each pixel, at least two or more different wavelength spectra of light, simultaneously and distinctively. Therefore, with the imaging element 36, the processing circuit 16 acquires the diffracted light D, resulting in acquisition of an image. That is, the processing circuit 16 controls the imaging element 36 to acquire an image, and acquires, based on the image acquired by the imaging element 36, information regarding the target T different from the solvent S, in a noncontact manner. Exemplary information regarding the target T includes the shape, contour, and size of the target T.

FIG. 4 illustrates an exemplary image acquired with the optical imaging apparatus 10 according to the present embodiment. FIG. 5 illustrates, as a comparative example, an exemplary image acquired without the aperture 34.

As illustrated in FIG. 4, the optical imaging apparatus 10 causes light reflective of structural information (contour) on the target T to reach the imaging element 36, and causes the imaging element 36 to acquire an image of the target T. In this case, since the parallel light P is blocked by the light-blocking region 34a of the aperture 34, no the parallel light P is incident into the imaging element 36, and the imaging element acquires the parallel light P as a black image. That is, an image acquirable by the imaging element 36 can indicate the contour or shape of the target T. Note that the size of an image acquirable by the imaging element 36 can be grasped, for example, based on the lens 32.

As illustrated in FIGS. 4 and 5, regardless of the presence or absence of the aperture 34, the acquired images are almost the same in contour/shape. The light-blocking region 34a of the aperture 34 according to the present embodiment is, for example, circular in shape, resulting in being axisymmetric about the optical axis L (z axis). The circular formation inhibits the anisotropy of light to enter the imaging element 36, namely, enables acquisition of an isotropic image. Thus, as illustrated in FIG. 4, an image to be acquired with the imaging element 36 of the optical imaging apparatus 10 according to the present embodiment can have a clear contour with less image distortion. Thus, it can be said that the image acquired with the optical imaging apparatus 10 according to the present embodiment illustrated in FIG. 4 indicates the acquisition of an image suppressed in distortion.

The image, as a comparative example, illustrated in FIG. 5 has an unclear boundary between the solvent S and the contour of the target T because of the acquisition of information regarding the solvent S together with the information regarding the target T. In contrast to this, the image illustrated in FIG. 4 has a clear boundary between the solvent S and the contour of the target T. Thus, it can be said that the image illustrated in FIG. 4 indicates the region varying in concentration ratio between the solvent S and the target T in the solvent S.

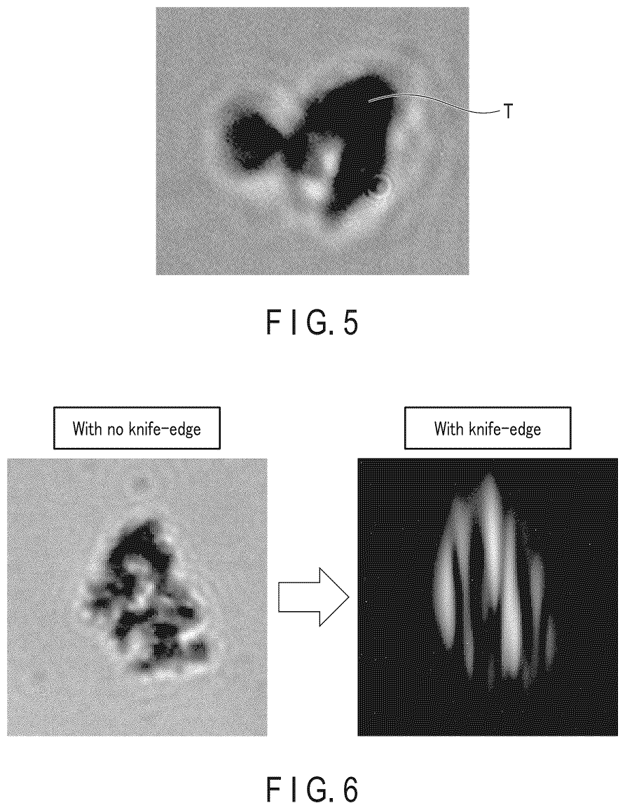

FIG. 6 illustrates, side by side, an exemplary image (with no knife-edge) acquired with the optical imaging apparatus 10, from which the aperture 34 is removed, according to the present embodiment and an exemplary image (with a knife-edge) acquired with a knife-edge for use in a typical Schlieren method, instead of the aperture 34 of the optical imaging apparatus 10 according to the present embodiment. The respective subjects in the images illustrated in FIG. 6 are the same. In comparison to the subject in the image on the left side in FIG. 6, the subject in the image on the right side in FIG. 6 has its contour extending in the direction of protrusion of the edge. In contrast to this, the image acquired with the optical imaging apparatus 10 according to the present embodiment (refer to FIG. 4) indicates that the contour is retained without any change, in comparison to the image acquired with the Schlieren method on the right side in FIG. 6.

Therefore, based on optical imaging with the aperture 34 that allows, in a case where the inspection object O is irradiated with the parallel light P including at least two or more different wavelength spectra of light, passage of the diffracted light D based diffraction due to passage of the parallel light P through the target T in the solvent S of the inspection object O and blocks the parallel light P having passed through the solvent S, the processing circuit 16 controls the imaging element 36 to acquire, as a color image, an image regarding the target T separated from an image regarding the solvent S. Thus, the optical imaging apparatus 10 enables acquisition of a color image as the information regarding the target T separated from the information regarding the solvent S, with control of the imaging element 36.

As described above, when the inspection object O is irradiated with the parallel light P, the parallel light P having passed through the receptacle R and the solvent S without irradiating the target T reaches, through the lens 32, the light-blocking region 34a of the aperture 34 at the focal point of the lens 32 and thus does not reach the imaging element 36. Thus, the imaging element 36 acquires the diffracted light D. Therefore, it can be said that an image acquirable with the optical imaging apparatus 10 according to the present embodiment is reflective of the structural information on the target T in the solvent S.

Note that the angle θ of diffraction of the diffracted light D varies depending on wavelengths. In general, a longer wavelength of light can cause a larger angle θ of diffraction. In the present embodiment, the angle θ of diffraction can vary between blue light at 450 nm and red light at 650 nm. The imaging element 36 acquires, at each pixel, at least two or more different wavelength spectra of light, simultaneously and distinctively. Therefore, the optical imaging apparatus 10 according to the present embodiment enables, with the imaging element 36, acquisition of the wavelength dependence of directional distribution of the diffracted light D due to the target T in the solvent S (image with color).

As above, an image acquirable by the imaging element 36 is reflective of information regarding the wavelength dependence of directional distribution of the diffracted light D and the structural information on the target T. The processing circuit 16 according to the present embodiment controls the light source 22 of the illuminator 12 to emit the parallel light P in a particular direction and controls the imaging element 36 to acquire the wavelength dependence of directional distribution of the diffracted light D due to the target T in the solvent S and the structural information on the target T through the imaging element 36.

In this case, the processing circuit 16 of the optical imaging apparatus 10 blocks and cuts, with the light-blocking region 34a of the aperture 34, the information regarding the solvent S and acquires, as an image, the information regarding the target T different from the solvent S. That is, the processing circuit 16 controls the imaging element 36 to acquire an image and then acquires the information regarding the target T different from the solvent S, based on the image acquired by the imaging element 36. Exemplary information regarding the target T includes the shape, contour, and size of the target T.

For example, the information regarding the target T includes a distribution of information related to a physical-property or physical-property-value. The information regarding the target T to be acquired with the imaging element 36 excludes the information regarding the solvent S. Thus, the processing circuit 16 can acquire or estimate the distribution of the information related to the physical-property of the target T different from the solvent S, due to the presence of the target T in the solvent S, by comparing, at each pixel of the image acquired by the imaging element 36, the intensities of two or more different wavelength spectra of light. The processing circuit 16 can estimate, as the information related to the physical-property of the target T, the concentration ratio of the target T in the solvent S. That is, an example of the information related to the physical-property is the concentration ratio of the target T in the solvent S. Due to acquisition of an image as the shape of the target T, the size of the target T can be grasped, so that the area (surface area) of the target T can be estimated. The optical imaging apparatus 10 can estimate the volume of the target T, based on integration of the area (surface area) of the target T.

### Application Example

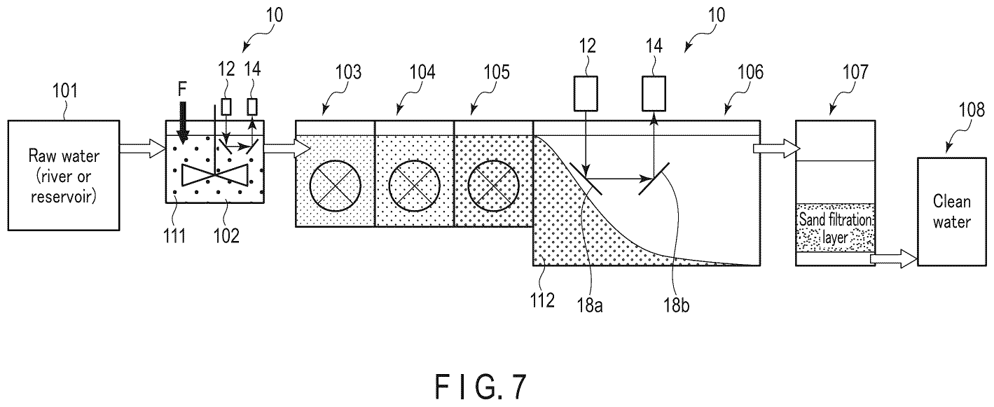

An application example of the optical imaging apparatus 10 according to the present embodiment will be described with FIGS. 7 to 15. In the present embodiment, a substance/object to be dealt with in a water treatment process corresponds to the target T. As the inspection object O, water corresponds to the solvent S and a floc due to flocculation in water treatment corresponds to the target T.

FIG. 7 illustrates a water treatment system 100.

As illustrated in FIG. 7, the water treatment system 100 includes a water source 101, such as a river or a reservoir, a mixing basin 102, flocculation basins (slow agitation basins) 103, 104, and 105, a sedimentation basin 106, a filtration basin 107, and a clean water reservoir 108. Water to be treated in the water source 101 moves from the water source 101 to the mixing basin 102, the flocculation basins (slow agitation basins) 103, 104, and 105, and the sedimentation basin 106 in this order.

In water treatment, colloidal suspended substances (suspended particles) flocculate due to injection of flocculant F, and the suspended substances lead to sedimentation in the sedimentation basin 106 to be described below. Such a settled flocculated substance has its colloidal particles, as the nucleus, to which gel adheres and is referred to as a floc 112.

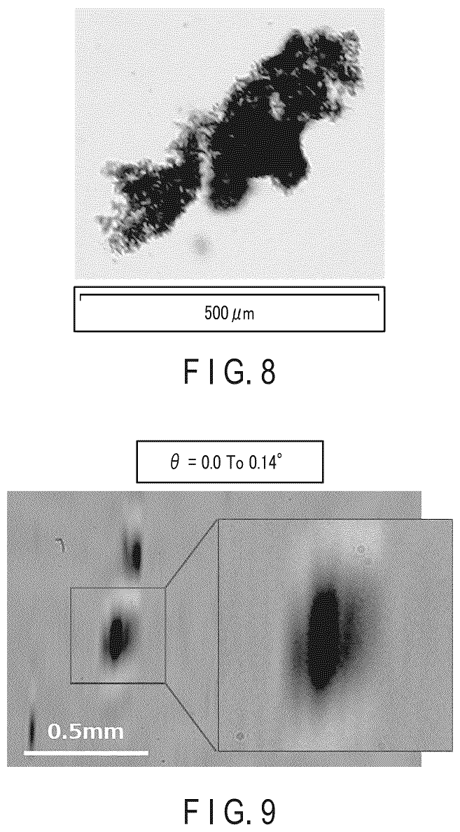

The water to be treated in the water source 101 includes, for example, sand and shingle in addition to water. The mixing basin 102 stores the water to be treated acquired from the water source 101. Floc is formed by injecting the flocculant F into the water to be treated in the mixing basin 102, stirring the water to be treated, and flocculating suspended particles (suspended substances) 111, for example, as illustrated in FIG. 8. The floc 112 illustrated in FIG. 8 was made with polyaluminum chloride (PACT) as the flocculant F and generally widely-used kaolin as a simulated substance for the suspended particles 111. Water was used as the solvent S.

The flocculation basins 103, 104, and 105 illustrated in FIG. 7 make flocs 112 flocculate gradually and coarse gradually by mixing the flocculant F. The sedimentation basin 106 causes the coarse flocs 112 to settle. Thus, the water being treated with a reduction in the quantity of suspended particles is located as a supernatant liquid above the flocs 112. The filtration basin 107 makes the water being treated that is the supernatant liquid, treated water, for example, by filtering with a sand filtration layer, and then discharges the treated water as clean water to the clean water reservoir 108. After the water being treated that is the supernatant liquid is discharged to the filtration basin 107, as necessary, the flocs 112 having settled in the sedimentation basin 106 are discharged from the sedimentation basin 106.

Setting the rate of injection of the flocculant F is an important operation having effect on the characteristics of performance of the following sedimentation and filtration processes or the quantity of generation of sludge. Thus, mainly, used are a method of setting the rate of injection manually based on operator's judgment responsive to the condition of performance and a method of changing the rate of injection depending on the turbidity of raw water. However, in order to favorably retain the state of flocs 112 generated after injection of the flocculant, the rate of injection of the flocculant requires adjusting, depending on the condition of quality of raw water, in some cases, leading to a burden to the operator. The agglomerate of a floc 112 is weak in coupling and collapses easily, and furthermore its gel is transparent. Thus, information acquirable by typical camera shooting is limited.

A parameter closely related to formation and sedimentation of a floc 112 is the concentration ratio ρ of the floc 112. The concentration ratio ρ herein corresponds to the ratio between the flocculant F and the suspended particles 111 included in the floc 112. The concentration ratio ρ is defined as follows:

ρ=(flocculant concentration [mg/L])/(suspended substance concentration [mg/L]).

Note that there has been no method enabling noncontact and prompt measurement or acquisition of the concentration ratio ρ of the floc 112.

In the water treatment system 100 according to the present embodiment, for example, as illustrated in FIG. 7, optical imaging apparatuses 10 are disposed. Referring to FIG. 7, the optical imaging apparatuses 10 are disposed to the mixing basin 102 and the sedimentation basin 106. However, for example, optical imaging apparatuses 10 may be disposed to the flocculation basins 103, 104, and 105. Preferably, the optical imaging apparatus 10 is disposed to at least one of the mixing basin 102 and the flocculation basin 103 as upstream as possible in a water treatment process. This is because the state of formation of a floc 112 can be grasped at an early stage during water treatment and, as necessary, required treatment can be performed easily during the water treatment.

In the mixing basin 102 and the sedimentation basin 106 illustrated in FIG. 7, the solvent S is, for example, water and the target T is, for example, a floc 112 including suspended particles 111. Note that, preferably, the illuminator 12 and the imaging portion 14 of each optical imaging apparatus 10 are disposed outside the water being treated instead of in the water being treated (solution). Meanwhile, reflective mirrors 18a and 18b for illuminating light are disposed in the water being treated in the mixing basin 102 or the like (refer to the sedimentation basin 106 in FIG. 7). Thus, each optical imaging apparatus 10 has an optical path having an appropriate length for illuminating light in the solvent S including the target T. In this case, because the mixing basin 102 has an opening on its upper side, the illuminator 12 and the imaging portion 14 of the optical imaging apparatus 10 can be appropriately installed. Thus, the receptacle R that is, for example, transparent for the inspection object O illustrated in FIGS. 1 and 3 is not required. Referring to FIG. 7, exemplarily, each optical imaging apparatus 10 has an optical path regulated along the drawing. However, for example, each optical imaging apparatus 10 may have an optical path orthogonal to the drawing. Referring to FIG. 7, exemplarily, the optical path is regulated with two mirrors 18a and 18b. However, a single mirror or three or more mirrors may be used. Each optical imaging apparatus 10 is disposed such that the target T can be observed on a desired optical path.

As described above, in a case where the target T in the solvent S has a structure equivalent to a level of wavelength of light, as illustrated in FIG. 3, if the parallel light P, with which the inspection object O is irradiated, hits the target T in the solvent S, diffraction of light occurs due to the target T. With the optical imaging apparatus 10 according to the present embodiment, many images of the inspection object O in the water treatment system 100 were acquired and then the angle θ of diffraction to the target T was obtained experimentally.

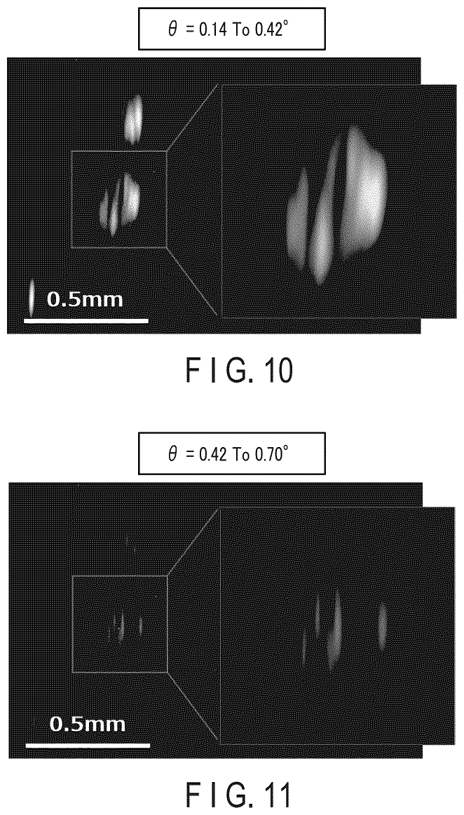

In the water treatment system 100, with such a floc 112 as illustrated in FIG. 8 as the target T, many images were acquired by the optical imaging apparatus 10 according to the present embodiment, and the angle θ of diffraction (0≤θ≤0.70°) was verified experimentally. FIG. 9 illustrates an exemplary image with the angle θ of diffraction satisfying the following condition: 0.0°≤θ<0.14°. FIG. 10 illustrates an exemplary image with the angle θ of diffraction satisfying the following condition: 0.14°≤θ<0.42°. FIG. 11 illustrates an exemplary image with the angle θ of diffraction satisfying the following condition: 0.42°≤θ<0.70°.

The optical imaging apparatus 10 disposed in the water treatment system 100 according to the present embodiment blocks, with the light-blocking region 34a, the parallel light P. Thus, it was found that, in a case where the optical imaging apparatus 10 according to the present embodiment is applied to the mixing basin 102 of the water treatment system 100, the angle θ of diffraction satisfying the following condition: 0.14°≤θ≤0.70° is preferable.

As above, in a case where the range of the angle θ of diffraction to the target T is known, f×tan θ is out of the size of the light-blocking region 34a, where f represents the focal length of the lens 32. Thus, the radius of the upper limit of the size of the light-blocking region 34a is f×tan θ. Due to such experiments, it was found that the light-blocking region 34a of the aperture 34 in the optical imaging apparatus 10 according to the present embodiment is preferably circular and its diameter is preferably 0.5 mm to the target T, resulting in adoption of this configuration. The aperture 34 is formed by forming a carbon with a diameter of 0.5 mm as the light-blocking region 34a and embedding the carbon in a thin and transparent glass plate with a thickness of 0.5 mm.

Therefore, the upper limit of the size of the light-blocking region 34a of the optical imaging apparatus 10 is set based on the focal length of the lens 32 and the angle θ of diffraction, and the lower limit thereof is set based on the size of projection of the light source 22 onto the light-blocking region 34a.

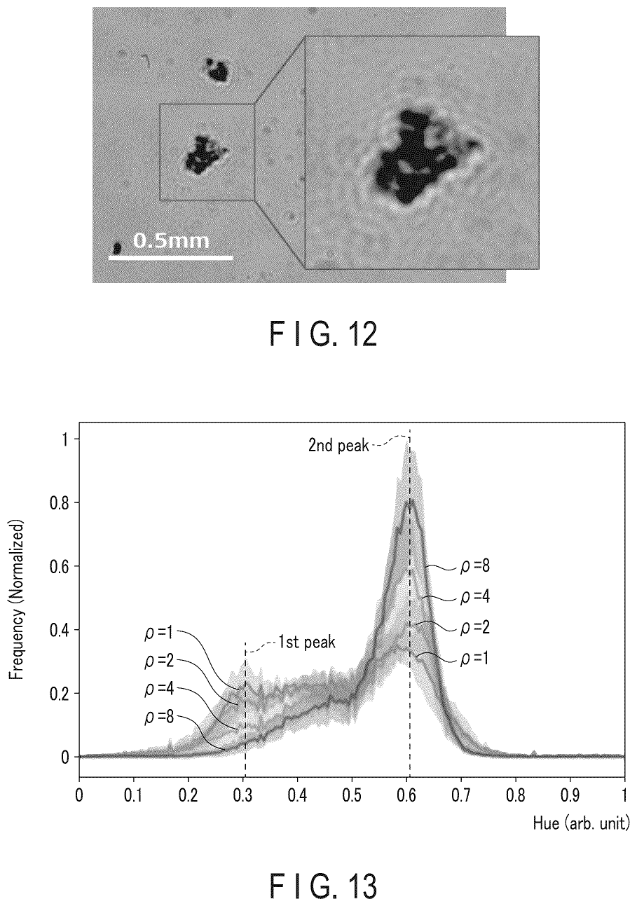

Note that FIG. 12 illustrates an exemplary image (solvent and target) acquired with no aperture 34 as a comparative example to the images illustrated in FIGS. 9 to 11. The image illustrated in FIG. 12 has an unclear boundary between the solvent and the target in pixel value.

Next, processing of estimating information related to a physical property of the target T with the optical imaging apparatus 10 applied to the water treatment system 100 will be described.

Various flocs 112 were acquired with the optical imaging apparatus 10. Information regarding the target was acquired as an image with the processing circuit 16. In addition, with a focus on the wavelength dependence of the acquired image, namely, the color of the image, the floc 112 was analyzed. The processing circuit 16 according to the present embodiment compares, at each pixel of the image acquired by the imaging element 36, the intensities of two or more different wavelength spectra of light and outputs the distribution of the information related to the physical-property of the target T. An exemplary of the distribution of the information related to the physical-property can be at least one of the density or density correlation value, material, refractive index, temperature, and distortion regarding the target T.

In the present embodiment, various flocs 112 different in concentration ratio ρ were each prepared, as the target T of the inspection object O, in the mixing basin 102 of the water treatment system 100. Here, flocs 112 of which the concentration ratio ρ are 1, 2, 4, and 8 were prepared. Then, a color image of the target T of each inspection object O was acquired by the optical imaging apparatus 10 according to the present embodiment disposed to the mixing basin 102 of the water treatment system 100. From the acquired color images, respective hue histograms were generated. FIG. 13 illustrates the results thereof.

The horizontal axis of the graph in FIG. 13 represents hue. Referring to FIG. 13, a larger value of hue on the horizontal axis is closer to purple shorter in wavelength, and a smaller value of hue on the horizontal axis is closer to red longer in wavelength. The vertical axis of the graph in FIG. 13 represents, as frequency ratio, the number of pixels as a function of hue. Expediently, the graphs were each normalized. It can be seen that each histogram illustrated in FIG. 13 has peaks at a hue of approximately 0.3 and a hue of approximately 0.6. Then, as illustrated in FIG. 13, it can be seen that the height of each peak varies in accordance with the concentration ratio ρ of the floc 112. For example, an increase in concentration ratio ρ caused a decrease in frequency at hues of 0.22 to 0.27 near the first peak. In contrast, an increase in concentration ratio ρ caused an increase in frequency at hues of 0.58 to 0.63 near the second peak.

Thus, the average (hues of 0.22 to 0.27) near the first peak and the average (hues of 0.58 to 0.63) near the second peak were each calculated, and then the ratio γ therebetween was defined as follows:

γ=(average of hues of 0.58 to 0.63)/(average of hues of 0.22 to 0.27).

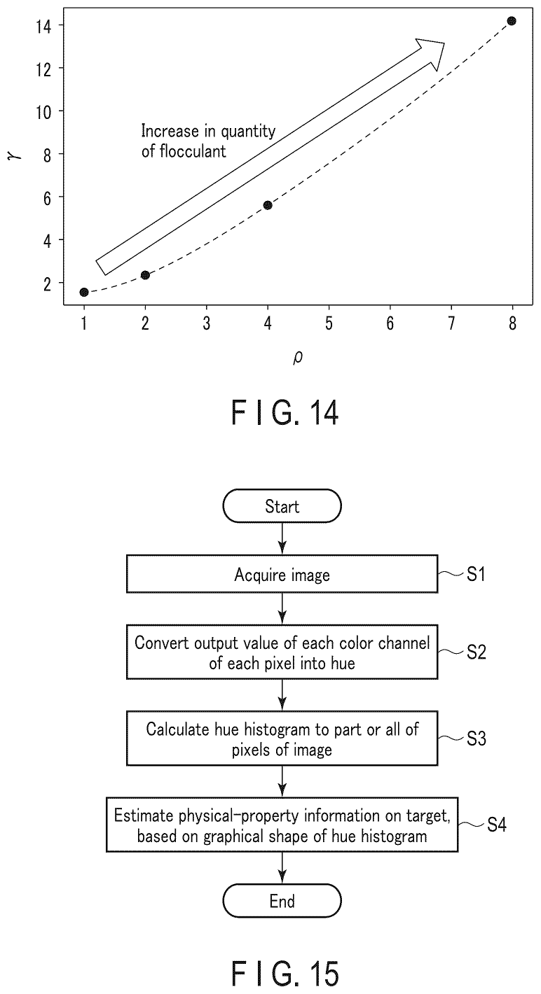

Then, γ was calculated at the concentration ratio ρ of each floc 112 for comparison. That is, at each pixel of the image acquired by the imaging element 36, the intensities of two different wavelength spectra of light were compared. As illustrated in FIG. 14, it was found that γ increases along with an increase in the quantity of the flocculant F. Thus, the optical imaging apparatus 10 according to the present embodiment and the analytical method with γ in combination enable estimation of the distribution of the information related to the physical-property of the target T, such as the concentration ratio ρ of a floc 112.

A flow of processing of estimating a distribution of information related to a physical-property of the target T, such as the concentration ratio ρ of a floc 112, with the optical imaging apparatus 10 applied to the water treatment system 100 will be described with FIG. 15. Note that the relationship between the concentration ratio ρ and γ illustrated in FIG. 14 is stored in advance in an auxiliary storage device.

The processing circuit 16 first causes the imaging element 36 to capture the inspection object O including the target T in the solvent S, and to acquire an image (step S1).

Based on the image of the target T acquired by the capturing with the imaging element 36, the processing circuit 16 converts the output value of each pixel included in the image of the target T into hue (step S2).

Furthermore, for processing to the desired image, the processing circuit 16 calculates a hue histogram to part or all of the pixels of the image (step S3).

The processing circuit 16 calculates γ described above from the hue histogram. That is, the processing circuit 16 compares the intensities of two different wavelength spectra of light at each pixel in the entirety or part of the image captured by the imaging element 36, and acquires information regarding the target T different from the solvent S. Then, the processing circuit 16 estimates the concentration ratio ρ corresponding to γ stored in the auxiliary storage device (step S4).

As above, for example, estimation of the concentration ratio ρ of a floc 112 in the mixing basin 102 enables estimation of an additionally required quantity of flocculant F in the water treatment system 100. The concentration ratio ρ varies from hour to hour due to the influence of nature, such as the rise of a river, muddiness in a river, or the water storage level of a reservoir, based on rainfall. Based on the concentration ratio ρ measured with the optical imaging apparatus 10 according to the present embodiment, a required quantity of flocculant F can be determined in real time.

Note that, for example, with the optical imaging apparatus 10 having the illuminator 12 and the imaging portion 14 constant in temperature, a change in the temperature of the target T causes a change in the refractive index of the target T. Thus, the optical imaging apparatus 10 compares between the intensities of two or more different wavelength spectra of light, and estimates the temperature or refractive index of the target T, namely, estimates a physical-property distribution regarding the target T.

Similarly, a change in the temperature of the target T can cause a change in the density correlation value or the density of the target T. The density correlation value or the density corresponds to the concentration ratio. A change in the concentration ratio of the target T causes a change in the temperature of the target T. Thus, the optical imaging apparatus 10 compares between intensities of two or more different wavelength spectra of light, and estimates the temperature or density correlation value of the target T, namely, estimates a physical-property distribution regarding the target T, or a distribution of information related to a physical-property of the target T.

Examples of the information regarding the target T different from the solvent S, described above, include the shape, density correlation value or density, volume, material, weight, refractive index, and the concentration ratio of the target T. As above, the optical imaging apparatus 10 according to the present embodiment enables noncontact acquisition of information according to a physical property of the target T in the solvent S, formerly difficult to measure or acquire promptly in a noncontact manner. Processing with the processing circuit 16 enables comparison between the intensities of two or more different wavelength spectra of light at each pixel of the image captured by the imaging element 36 and estimation of a distribution of information related to a physical-property of the target T different from the solvent S.

As above, according to the present embodiment, provided can be the optical imaging apparatus 10, the processing circuit (processing apparatus) 16, the optical imaging method, and the non-transitory storage medium storing the optical imaging program that enable noncontact acquisition of information according to the target T in the solvent S.

In the above example, the application of the optical imaging apparatus 10 according to the present embodiment to the mixing basin 102 or the like of the water treatment system 100 has been given. The optical imaging apparatus 10 according to the present embodiment can be used in various fields, such as the medical field and the marine field.

For example, in the medical field, the optical imaging apparatus 10 according to the present embodiment can be used for acquisition of information regarding tissue in a cell membrane. The optical imaging apparatus 10 according to the present embodiment enables, for example, acquisition of an image of the structure of a transparent cell. Examples of the information regarding the target T different from the solvent S include cytoplasm, a nucleus, and a mitochondrion. Then, based on an image including the information regarding the target T, acquired with the processing circuit 16, at least one of the shape, density correlation value or density, concentration ratio, volume, material, weight, refractive index, temperature, and distortion of the tissue or nucleus in the cell can be estimated.

In the marine field, the optical imaging apparatus 10 according to the present embodiment is installed on an ocean, so that an image of the shape of the target T, such as a microplastic, can be acquired. Based on the acquired image including the information regarding the target T, at least one of the shape, density correlation value or density, concentration ratio, volume, material, and weight of the target T can be estimated. The optical imaging apparatus 10 according to the present embodiment enables, for example, visualization of the degree of pollution of a particular area in the sea in real time.

In addition, for example, in laser peening to improve a target material with emission of laser light into the water, a scattered object from the target material can be acquired as the target T and then be analyzed. Furthermore, occurrence of cavitation in the laser peeing can be acquired and then be analyzed. Thus, the optical imaging apparatus 10 according to the present embodiment enables, for example, capturing of a phenomenon or mechanism due to the laser peening.

In the embodiment described above, water is exemplarily used as the solvent S. However, an appropriate solvent different from such water can be used.

In the present embodiment, given has been the example in which the light-blocking region 34a is formed by disposing carbon to glass for the aperture 34. For example, the light-blocking region 34a may be achieved with an electronic shutter. In this case, the target T having various angles θ of diffraction can be shot, as described above, with a single aperture.

The optical imaging apparatus 10, the processing circuit (processing apparatus) 16, the optical imaging method, and the non-transitory storage medium storing the optical imaging program, according to at least one of the embodiments described above, enable noncontact acquisition of the information according to the target T in the solvent S.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would 5 fall within the scope and spirit of the inventions.

What is claimed is:

1. An optical imaging apparatus comprising:
an illuminator that is configured to emit parallel light including at least two or more different wavelength spectra of light;
a lens into which light is incident through an inspection object provided where the parallel light from the illuminator reaches,
the inspection object including a solvent and a target in the solvent serving as a substance different from the solvent,
the light having passed through the solvent and the target, and/or through the solvent;
an aperture disposed on a focal plane of the lens, the aperture including:
a passage region allowing passage of diffracted light in a direction different from a direction of the parallel light due to the target from the parallel light from the illuminator, and
a light-blocking region blocking the parallel light having passed through the solvent; and
an imaging element that is configured to acquire, at each pixel, in response to arrival of the diffracted light based on diffraction due to the target at an imaging plane through the passage region, the at least two or more different wavelength spectra of light, simultaneously and distinctively.

2. The optical imaging apparatus according to claim 1, further comprising a processing apparatus that is configured to acquire, based on an image acquired by the imaging element, information regarding the target different from the solvent.

3. The optical imaging apparatus according to claim 2, wherein:
the processing apparatus is configured to:
compare, at each pixel of the image acquired by the imaging element, intensities of the at least two or more different wavelength spectra of light, and
estimate a distribution of information related to a physical-property of the target different from the solvent.

4. The optical imaging apparatus according to claim 3, wherein:
when the processing apparatus estimates the distribution of the information related to the physical-property of the target, the processing apparatus is configured to:
convert an output value of each pixel of the image into hue, and
calculate a hue histogram to part or all of the pixels of the image.

5. The optical imaging apparatus according to claim 3, wherein the distribution of the information related to the physical-property of the target corresponds to at least one of density correlation value or density, concentration ratio, volume, material, weight, refractive index, temperature, and distortion regarding the target.

6. The optical imaging apparatus according to claim 1, wherein:
the light-blocking region is disposed at a focal position of the lens and is axisymmetric about an optical axis of the lens, and
a size of the light-blocking region is identical to or larger than a size of projection of light from a light source of the illuminator.

7. The optical imaging apparatus according to claim 6, wherein the light-blocking region has a circular shape.

8. The optical imaging apparatus according to claim 7, wherein
in a case where an angle θ of diffraction to the target is known, f×tan θ is larger than a size of a radius of the light-blocking region, where f represents a focal length of the lens.

9. A processing apparatus for use in optical acquiring of a target, the processing apparatus comprising a processor configured to:
control, in a case where an inspection object is irradiated with parallel light including at least two or more different wavelength spectra of light, an imaging element to acquire, as a color image, an image regarding the target in solvent separated from an image regarding the solvent,
based on optical imaging with an aperture that:
allows passage of diffracted light based on diffraction due to passage of the parallel light through the target of the inspection object, and
blocks the parallel light having passed through the solvent; and
acquire information regarding the target different from the solvent, based on the color image acquired by the imaging element.

10. The processing apparatus according to claim 9, wherein the processor is configured to:
compare, at each pixel of the color image, intensities of the at least two or more different wavelength spectra of light, and
estimate a distribution of information related to a physical-property of the target different from the solvent.

11. The processing apparatus according to claim 10, wherein
When the processor estimates the distribution of the information related to the physical-property of the target, the processor is configured to:
convert an output value of each pixel of the color image into hue, and
calculate a hue histogram to part or all of the pixels of the color image.

12. An optical imaging method for a target, the optical imaging method comprising:
acquiring, in a case where an inspection object is irradiated with parallel light including at least two or more different wavelength spectra of light, with an imaging element, as a color image, an image regarding the target in solvent separated from an image regarding the solvent,
based on optical imaging with an aperture that:
allows passage of diffracted light based on diffraction due to passage of the parallel light through the target of the inspection object, and
blocks the parallel light having passed through the solvent; and
acquiring information regarding the target different from the solvent, based on the color image acquired by the imaging element.

13. The optical imaging method according to claim 12, wherein
the acquiring of the information regarding the target includes:
comparing, at each pixel of the color image, intensities of the at least two or more different wavelength spectra of light, and
estimating a distribution of information related to a physical-property of the target different from the solvent.

14. The optical imaging method according to claim 13, wherein
the estimating of the distribution of the information related to the physical-property of the target includes:
converting an output value of each pixel of the color image into hue; and
calculating a hue histogram to part or all of the pixels of the color image.

15. A non-transitory storage medium storing an optical imaging program for a target, the optical imaging program causing a computer to perform:
acquiring, in a case where an inspection object is irradiated with parallel light including at least two or more different wavelength spectra of light, with an imaging element, as a color image, an image regarding the target in solvent separated from an image regarding the solvent,
based on optical imaging with an aperture that:
allows passage of diffracted light based on diffraction due to passage of the parallel light through the target of the inspection object, and
blocks the parallel light having passed through the solvent; and
acquiring information regarding the target different from the solvent, based on the color image acquired by the imaging element.
